(19) United States
(12) Patent Application Publication
(10) Pub. No.: US 2016/0128776 A1
(43) Pub. Date: May 12, 2016

(54) DIFFUSE REFLECTORS AND METHODS OF USE THEREOF

(71) Applicant: John Curtis Walling, Whitehouse Station, NJ (US)

(72) Inventor: John Curtis Walling, Whitehouse Station, NJ (US)

(21) Appl. No.: 14/938,777

(22) Filed: Nov. 11, 2015

Related U.S. Application Data
(60) Provisional application No. 62/078,003, filed on Nov. 11, 2014.

(51) Int. Cl. A61B 18/20; H01S 3/08; H01S 3/16

(52) U.S. Cl. CPC A61B 18/203; H01S 3/1643; H01S 3/1611; H01S 3/1603; H01S 3/1633; H01S 3/08059; A61B 2018/2035; A61B 2018/0047

(57) ABSTRACT

In some embodiments, the present invention provides for a laser pump chamber, including: at least one laser gain medium, at least one excitation source, and at least one diffuse reflector to direct and redirect an emission from the excitation source into the laser gain medium, wherein the at least one diffuse reflector is made from a diffuse reflector material comprising at least one of: 1) white quartz and 2) BaSO4.

### RELATED APPLICATIONS

This application claims the priority of U.S. provisional patent application No. 62/078,003; filed Nov. 11, 2014; entitled “DIFFUSE REFLECTORS AND METHODS OF USE THEREOF,” which is incorporated herein by reference in its entirety for all purposes.

### FIELD OF INVENTION

In some embodiments, the present invention is related to diffuse reflectors for solid state lasers and methods of use thereof.

### BACKGROUND OF THE INVENTION

For instance, a solid-state laser is a laser that uses a gain medium that is a solid, rather than a liquid such as in dye lasers or a gas as in gas lasers.

### SUMMARY OF THE INVENTION

In some embodiments, the present invention provides for a laser pump chamber, including: at least one laser gain medium, at least one excitation source, and at least one diffuse reflector to direct and redirect an emission from the excitation source into the laser gain medium, wherein the at least one diffuse reflector is made from a diffuse reflector material comprising at least one of: 1) white quartz and 2) BaSO4.

In some embodiments, the diffuse reflector material comprises the white quartz and the BaSO4. In some embodiments, the at least one laser gain medium includes at least one of Ce:Nd:YAG, Nd:YAG, and/or alexandarite.

In some embodiments, the present invention provides, including a step of utilizing the laser pump chamber of the present invention for of at least one of: 1) at least one medical procedure, and 2) at least one dermatological procedure. In some embodiments, the at least one dermatological procedure is at least one of: i) hair removal, ii) a tattoo removal, and iii) a skin resurfacing.

### BRIEF DESCRIPTION OF THE DRAWINGS

The present invention can be further explained with reference to the attached drawings, wherein like structures are referred to by like numerals throughout the several views. The drawings shown are not necessarily to scale, with emphasis instead generally being placed upon illustrating the principles of the present invention. Therefore, specific structural and functional details disclosed herein are not to be interpreted as limiting, but merely as a representative basis for teaching one skilled in the art to variously employ the present invention.

FIG. 1 a screenshot, showing an embodiment of the present invention, illustrating a quartz reflective coating in accordance with the present invention.

FIG. 2 a screenshot, showing an embodiment of the present invention, illustrating a graphical representation of the reflective coating.

FIG. 3 a screenshot, showing a tube that can be used in an embodiment of the present invention.

FIGS. 4A-4H are screenshots, showing dimensions of tubes that can be used in embodiments of the present invention.

FIG. 5 a screenshot, showing a photograph of an operational white quartz (HOD-500EX) pump chamber, using and extended version of the reflector of FIG. 4B in accordance with the present invention.

FIG. 6 a screenshot, showing a schematic of a pump cavity configuration illustrating the principle of balance of geometry in accordance with the present invention.

The figures constitute a part of this specification and include illustrative embodiments of the present invention and illustrate various objects and features thereof. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

### DETAILED DESCRIPTION OF THE INVENTION

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

In some embodiments, the inventive laser devices of the present invention can include a diffuse reflector component consisting of “white quartz.” As used herein, the term “white quartz” is defined as a form of ultrapure, quartz or silica, either crystalline or amorphous, that is perfused with a high density of tiny light-scattering gas bubbles, which gives the material essential properties sufficiently suitable for diffuse reflecting of light. In some embodiments, the white quartz can be used for the diffuse reflector in the pump chamber(s) of host-type solid state lasers such as, but not limited to, alexandrite, Ce:Nd:YAG, and/or Nd:YAG lasers, and driven (pumped) by light emitting sources, including but not limited to lamps, flash lamps and/or laser diodes. For example, the diffuse reflecting material in diffuse reflection type laser pump chambers directs or redirects light emission energy into the laser gain medium, where it is converted by laser action into the laser beam.

In some embodiments, the term “white quartz” can also be referred to as: fused silica, fused quartz, and crystalline quartz, a material occurring in nature. Typically, the molecular composition of such quartz is SiO2. In some embodiments, quartz's characteristics can be dependent on the way the molecules of SiO2 are ordered in the solid, their source, and purity. In some embodiments, the inventive devices of the present invention can utilize white quartz such as, but is not limited to, HOD-500EX (Heraeus Optical Diffuser; Heraeus Quarzglas, Hanau, Germany (Heraeus)) and HOD-500 (Heraeus). In some embodiments, Heraeus OM-100 opaque quartz glass can be used to correct a typical problem in design, where, e.g., o-ring seals are damaged by high intensity lamp emission that becomes trapped in glass tubes exposed to the interior of the pumping chamber, where the photon flux is high. For example, if the trapped emission is transmitted by internal reflection (a phenomenon herein referred to as “light piping”) to the o-ring seals conforming to the tube damaging the seals, the trapped emission can cause them to fail. In some embodiments, OM-100 tubes can be welded to the quartz glass tubes, extending them, which scatter the piped light, protecting the seal. In some embodiments, the opaque quartz glass can be HOD-500 (Heraeus).

In some embodiments, the inventive laser devices of the present invention can be used in lasers for medical and dermatological cosmetic applications, including but not limited to laser hair removal and/or tattoo removal, and/or skin resurfacing.

In some embodiments, the present invention relies on a principle that the scattering by the bubbles and/or the transmission properties of white quartz (e.g., highly purity) to make it in sufficient thickness highly efficient (e.g., the efficiency of white quartz as a diffuse reflector of light is comparable to that of powdered barium sulfate (BaSO4)) as a diffuse reflector of light, comparable to that of any other know material, solid or powder. However, the same reflection efficiency requires a greater thickness of either HOD-500 or HOD-500EX compared to BaSO4. To gain the advantage of the nonporous nature of white quartz, which BaSO4 does not have, the reflector can be made in two shells, the inner shell of white quartz, which also is where the o-ring seal is made, but where it is then protected by the white quartz eliminating the problem associated with its use, and the outer shell of powdered BaSO4, which can be contained separate from fluids in the pump chamber that would destroy its high diffuse-reflecting efficiency if absorbed by it.

In some embodiments, the white quartz may contain bubbles of smaller size (0.1 μm to 10 μm, preferably) and can be 10 to 1000 times higher density than that in HOD-500EX or HOD-500. Such size and density would enable to the white quartz to be effective in much reduced thicknesses, making it more effective in smaller pump chamber designs.

In some embodiments, the system of the present invention includes a pump chamber diffuse reflector, where the pump chamber reflector can be in two halves, that are in the form of two tubes each closed on one end with holes made through the ends to accommodate the lamp(s) and laser rod. In some embodiments, this two-halve design allows for the flow tubes, however designed, to be inserted before the two haves are joined at their open ends to create a pumping cavity surrounded by diffuse reflecting material, which contains the flow tubes mentioned above. For example, the two halves can be identical (or nearly so) and are connected and sealed together where they join. In addition, any other similarly suitable joining techniques can be utilized to create this seal.

In some embodiments, a characteristic of white quartz can include a fused surface impenetrable by water or other coolant, e.g., as a nonporous solid reflector. In some embodiments, white quartz, can be used in a laser pump chamber, and the white quartz can have the following properties: high purity, ultra-low absorptivity, high durability, non-porosity, high diffuse reflectivity, and be of a solid material (e.g., HOD-500EX).

In some embodiments, the inventive laser devices of the present invention include a diffuse reflecting ceramic composition(s) for a laser pump chamber. In some embodiments, the diffuse reflecting ceramic composition(s) can typically provide up to 4% efficiency when used, e.g., but not limited to, in Nd:YAG lasers. In some embodiments, the diffuse reflecting ceramic composition(s) utilized in the inventive laser devices of the present invention can be BaSO4 and/or white quartz, and can provide between 1-6% efficiency when used, e.g., but not limited to, in Nd:YAG lasers. In some embodiments, the inventive laser devices of the present invention can include, but is not limited to, a Nd:YAG or alexandarite flash lamp pumped laser. In some embodiments, the system includes, but is not limited to a Nd:YAG and/or alexandrite diode laser pumped laser.

In some embodiments, the inventive laser devices of the present invention include a high power BaSO4 pump chamber. In some embodiments, the inventive laser devices of the present invention include a pump chamber, where the pump chamber includes white quartz. In some embodiments, white quartz can comprise the reflector in a laser pump chamber, utilizing, for example, but not limited to, Nd:YAG, alexandrite, or other suitable gain material, as the active media. In some embodiments, the white quartz can be fused on the outside of a flow tube containing rod and lamps. In some embodiments, the inventive laser devices of the present invention achieve the result which is the same as, e.g., putting a hole through a block of white quartz, through which the flow tube fits. In some embodiments, the block of white quartz having a hole for the lamp and laser rod may not use a flow tube.

In some embodiments, the cross sectional shape of the flow tube, or hole through the reflector may be a race track configuration, or an ellipse or a circle, or other similar configuration, or one with additional baffles to help guide the coolant flow.

In some embodiments, the inventive laser devices of the present invention can be diode lasers which utilize the white quartz as a reflector for, in some cases, diode pumping alexandrite to make a diode pumped alexandrite laser.

In some embodiments, the inventive laser devices of the present invention utilize at least one diffuse reflector, where the diffuse reflector is solid (can be porous and/or nonporous), including ZrO2 powder having a high index of refraction together with a powder of a low index of refraction with or without a third powder of a matrix compound used to bind them. In some embodiments, the inventive laser devices of the present invention are required that parts of the the diffuse reflector must be highly transparent and may or may not be sintered together. The sintering, may involve the melting of the matrix compound in which the other components remain in their original crystalline form.

In some embodiments, the inventive laser devices of the present invention utilize the composition of the diffuse reflector that may be a mixture of powders of ZrO2 with BaSO4 (barium sulfate) having powder size in the range between 10 nm and 10 um. In some embodiments, the particles of ultrapure ZrO2 and/or the low index material can be in the range from 300 nm to 2000 nm. In some embodiments, the low index material may be ultrapure BaSO4, such as Eastman White Reflectance Standard (Eastman Kodak Company, Rochester, N.Y.)

In some embodiments, the inventive laser devices of the present invention utilize the reflector that can be molded using powdered SiO2 in particle size of 100 to 10000 nm that is packed around a solid model of the reflector, such that the model can be removed leaving a space wherein that can be filled with the reflector composition powders, and then put into a furnace, with or without an O2 gas perfusion to kept the powders well oxygenated during the heating process. For example, the temperature of the oven may rise to just begin to melt the BaSO4 and thus sinter the materials together, or to well above the melting point of the BaSO4, such that it becomes liquid in which the particles of Zr04 are suspended. In some embodiments, the furnace may be evacuated in advance of and during the heating cycle so that the air is removed from the reflector material which then forms a reflector without air bubbles. For example, nano particles of BaSO4 may be used to enable the material to be nonporous when the sold is formed.

In some embodiments, the inventive laser devices of the present invention utilize ZrO2 that can be in the form of small particle powders, in the range of diameters less than 10 μm and greater than 3 nm . In some embodiments, the inventive laser devices of the present invention utilize fused silica that is in the form of small particle powders and, both ZrO2 and fused silica can be mixed as powders together and combined with, e.g., but not limited to, 7056 alkali borosilicate (BS) glass (Corning) (7056). In some embodiments, the powders are allowed to be mixed before melting, softening the BS component in, e.g., a furnace. In some embodiments, the fused silica helps keep the ZrO2 particles dispersed. In some embodiments, when the BS component has a finer powder, the fused silica may not be required. Based on the exceptionally high index of refraction of ZrO2, 2.16, in relation to the other materials used with them, these reflectors, by design, are sufficiently efficient in thicknesses comparable to dry, powered BaSO4.

In some embodiments, the inventive laser devices of the present invention utilize the particles of high index ZrO2 which are dispersed and produce a high reflection in the low index media. In some embodiments, the inventive laser devices of the present invention utilize the relative concentration of ZrO2, 7056, SiO2, or any combination thereof (e.g., sufficiently equal proportions). In some embodiments, air can be trapped in the melt and could improve the characteristics of the system, where the improvement is based on the proportion of 7056 present. In some embodiments, a lower concentration of 7056 can result in more porosity. In some embodiments, the temperature and/or time of heating/cooling can impact the result of the reflector in accordance with the present invention.

In some embodiments, the inventive laser devices of the present invention utilize a YAG pump chamber that can include at least one diffuse reflector, where the diffuse reflector can be made from the white quartz and/or a ZrO2/borosilicate glass mixture. In some embodiments, an absorber can be placed at the ends of the reflector, where the absorber serves to protect the seals. In some embodiments, coolant seals can be placed on both ends, toward or at the back of a nonporous reflector where the light does not reach, thus protecting the seals from the flash lamp emission.

In some embodiments, the inventive laser devices of the present invention utilize are in form of an alexandrite diode pumped system, where the system includes a diffused reflector(s) to trap the power.

In some embodiments, the inventive laser devices of the present invention utilize a reflective composition of high purity and/or high reflectivity that can withstand high fluence of wavelengths from 225 nm to roughly 1 μm. In some embodiments, the reflective composition is white quartz (e.g., but not limited to, HOD-300) and/or fused silica having like high reflectivity properties (typically purer and less likely to solarize than, e.g., HOD-300). In some embodiments, the reflectivity of the reflective composition can be >99.9 over the range from 300 nm to 850 m. In some embodiments, the reflective composition can avoid deterioration over time from solarization and/or color center formation.

In some embodiments, white quartz can be prepared by combining two parts of white quartz, which can be combined by using epoxy, to make the exemplary reflector of the present invention. In some embodiments, a notch around the periphery of the combined white quartz part can provide a space for epoxy, where like parts are butted against each other end to end. In some embodiments, the surface of the material, e.g., white quartz, when porous can be flame polished. In some embodiments, when the surface of the material, e.g., white quartz, is nonporous, flame polishing may not be performed. In some embodiments, the joining surfaces are flat and cut substantially precisely normal to the axis of the hole, so the parts will fit closely together and line up properly.

In some embodiments, the inventive laser devices of the present invention utilize opaque quartz glass. In some embodiments, the opaque quartz glass can be OM-100. In some embodiments, the OM-100 can be used to substantially stop light piping in the glass (where, if the light is not substantially stopped, will cause, e.g., overheating of the O-rings used to seal the process chambers).

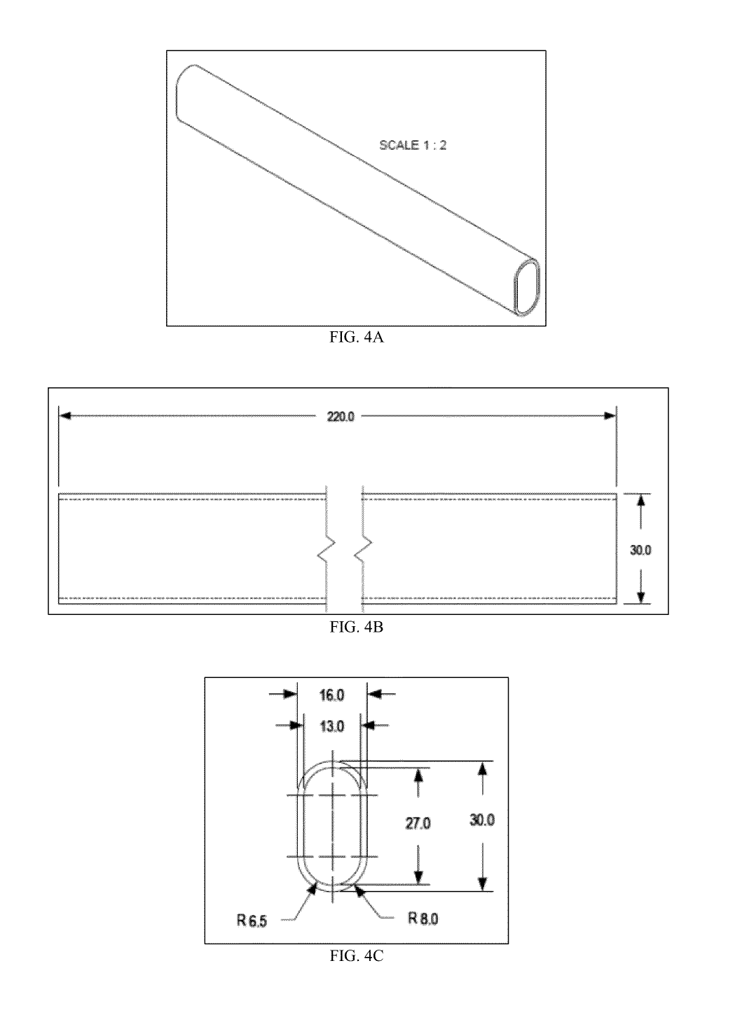

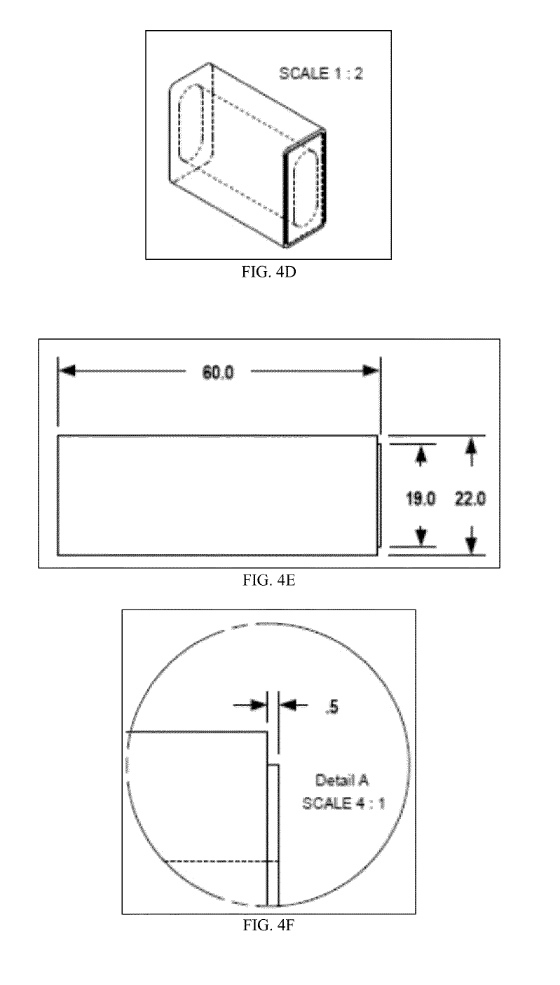

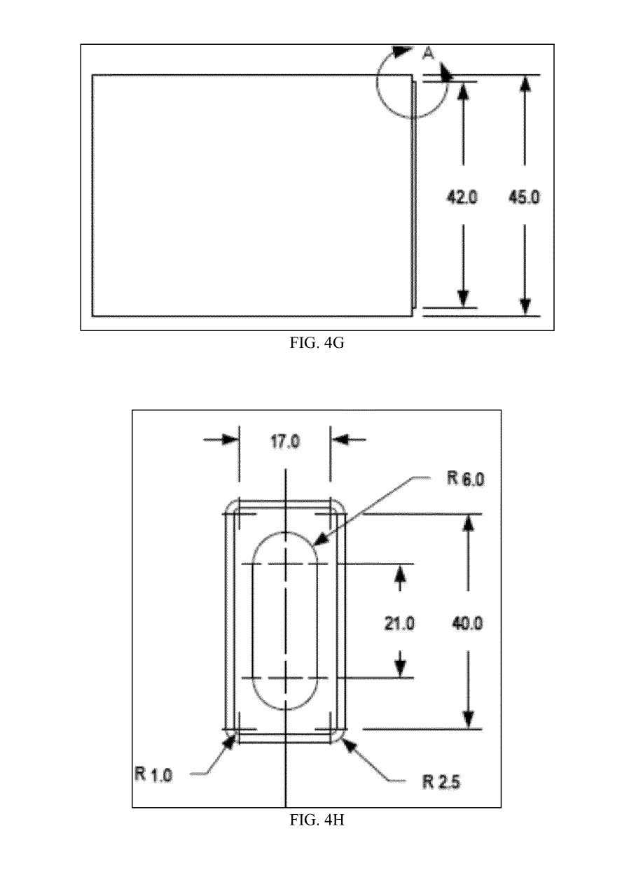

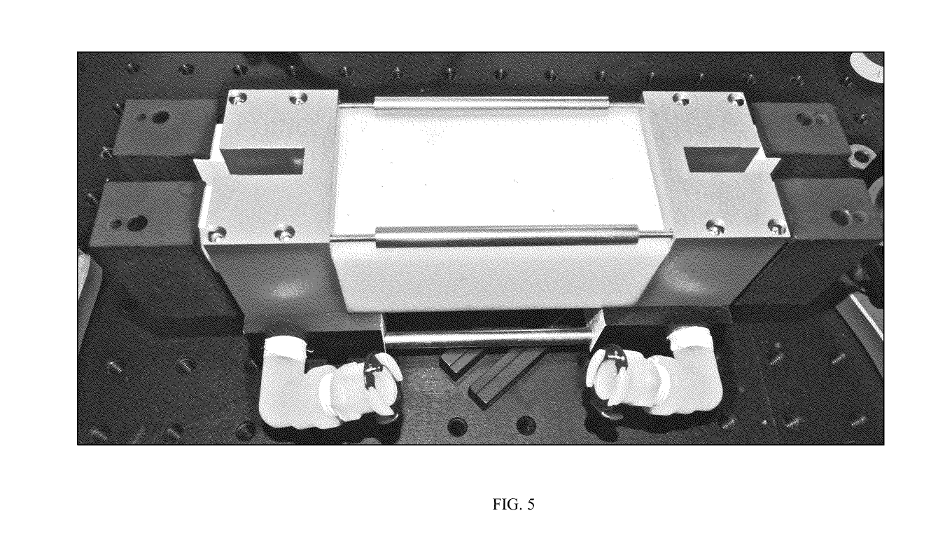

In some embodiments, the present system uses a reflective composition, where the reflective composition can be: white quartz HOD-500EX200 diffuse reflector/ “White Fused Silica Diffuse Reflector” (RD-HD-PTS-2397) (side 1: 60 mm, side 2: 45 mm, side 3: 22 mm), HOD-500EX® diffuse reflector/“white fused silica diffuse reflector” (side 1: 120 mm, side 2: 45 mm, side 3: 22 mm), HOD-300EX® diffuse reflector/white fused silica diffuse reflector (side 1: 120 mm, side 2: 45 mm, side 3: 22 mm), HOD-500EX® diffuse reflector/white fused silica diffuse reflector (side 1: 120 mm, side 2: 45 mm, side 3: 22 mm), or any combination thereof. In some embodiments, the holes need to be of 12 mm Inside diameter, Reflective Coating (e.g., Heraeus) substantially all the way around, and the QRC type coating can be touted as, e.g., but not limited to, only 80% reflective, e.g., the same as OM-100. In some embodiments, the reflectivity of Reflective Coating, can measure up to the 90th percentile. In some embodiments, reflective composition can have a dual lumen, quartz tube, measuring, e.g., 12 mm inside diameters, coated all around with, e.g., 3-8 mm of reflective coating, or any combination thereof. In some embodiments, the tube is an oval racetrack tube, of the dimensions specified in e.g., FIG. 4A and FIG. 4B. In some embodiments, the tube has a 3-4 mm reflective coating fused around the outside.

In some embodiments, the inventive laser devices of the present invention utilize an IR lamp with a highly reflective opaque quartz coating directly on the tube. In some embodiments, the IR lamp can be a single tube. In some embodiments, the IR lamp can be a twin tube.

In some embodiments, the inventive laser devices of the present invention utilize a (double doped) cerium + neodymium doped YAG, Ce:Nd:YAG, in which case the efficiency can be as much as 30% to 40% greater than Nd:YAG alone. Such Ce:Nd:YAG, material is available commercially for example from Castech Inc. (China).

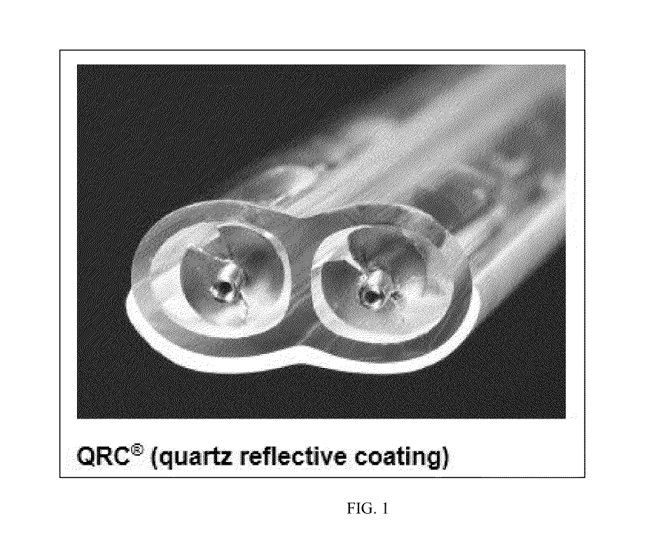

FIG. 1 shows an embodiment of the present invention, illustrating quartz reflective coating.

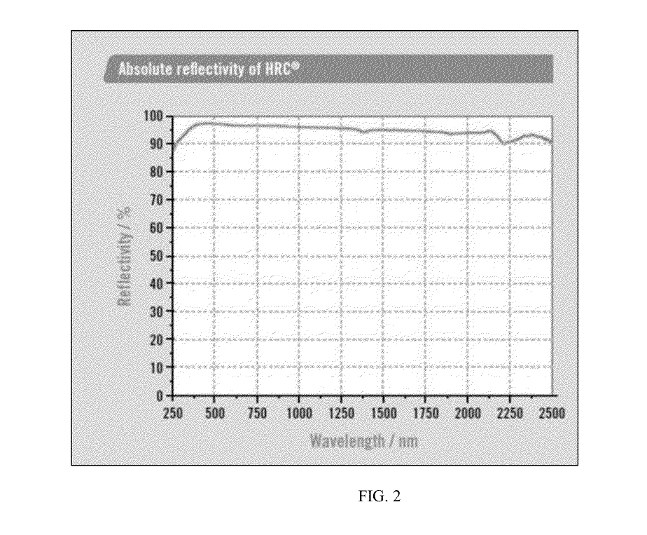

FIG. 2 shows an embodiment of the present invention, illustrating a graphical representation of the reflective coating.

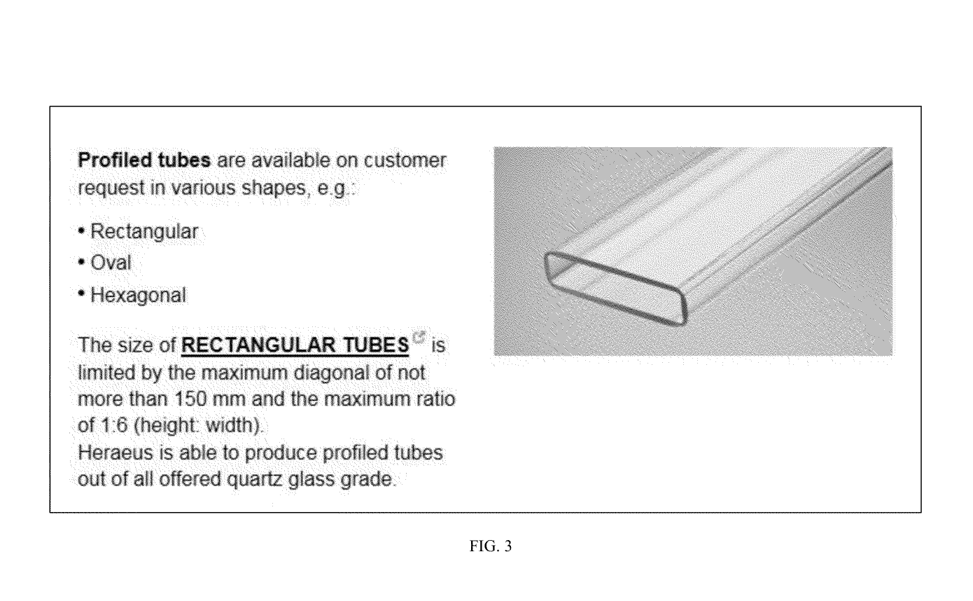

FIG. 3 shows a tube that can be used in an embodiment of the present invention.

FIGS. 4A-4C show dimensions of a tube that can be used in embodiments of the present invention.

FIGS. 4D-4H show dimensions of another tube that can be used in embodiments of the present invention.

FIG. 5 shows a photograph of an operational white quartz (HOD-500EX) pump chamber, using and extended version of the reflector of FIG. 4B.

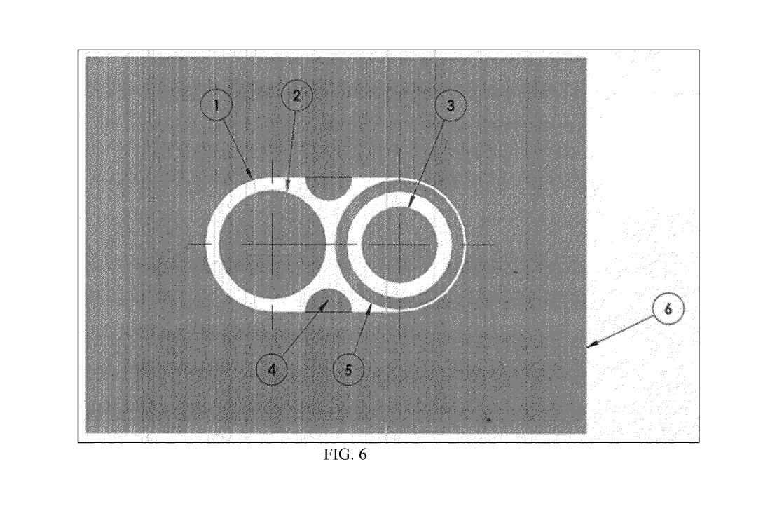

FIG. 6 shows a schematic of a pump cavity configuration illustrating the principle of balance of geometry. For example, an illustrative laser device of the present invention can include at least one laser rod (3), at least one defuse reflector (6) having a race track hole (1), an optional coolant flow baffle(s) (4), a rod flow tube(5), and a lamp having a lamp jacket outer diameter (2). For example, the clear spaces within the reflector (6) are where the coolant flows. For example, the baffles (4) are clear fused silica half rods used to improve coolant flow.

In some embodiments, the inventive laser devices of the present invention utilize a laser pump chamber that contains a pumping enclosure (also referred to herein as a cavity or pumping cavity), defined as the enclosed volume within the pump chamber where the lamp emission is transferred to the laser medium, e.g. a laser rod of Nd:YAG. For example, the pumping enclosure can be cooled by a coolant such filtered pure water or any other similarly suitable coolant which can circulate (flow) through it. In some embodiments, the inventive laser devices of the present invention can utilize different types of pumping enclosures for lamp excitation and for diode laser excitation of the active laser medium.

For example, a cross section of the pumping enclosure design for having one rod and one lamp can be configured to include, as shown, for example, in FIG. 6: a race track flow tube that is used to contain the rod and lamp and coolant, and this is inserted into a race track hole in a diffuse reflector. In some embodiments, if the reflector is nonporous, it can serve as the racetrack flow tube as well. In some embodiments, dimensions of the race track flow tube, the lamp diameter, the laser rod diameter, and the rod flow tube can be determine by the combined fit of the components at the expense of completely uniform, cylindrically symmetric flow of coolant over the lamp. For example, the laser rod requires such flow to maintain alignment of the beam and so a circular cross section flow tube around the circular-cross section laser rod is required. In some embodiments, the flow over the lamp need not be so uniform, just sufficient in speed over the surface. For example, the lamp jacket and the rod flow tube outer diameter can be made to fit within the race track flow tube to provide sufficient flow over the lamp, but have a sufficiently limited flow over the outside of the somewhat larger diameter rod flow tube, and sufficiently uniform flow over the laser rod. For example, the relatively larger rod flow tube allows maximally sized laser rod diameter for sufficient optical transfer of energy from the lamp lumen to laser rod, achieving higher overall efficiency.

The advantage of this configuration also includes the use of only one (inexpensive) round flow tube. If the diffuse reflector can itself serve as the race track flow tube, by being nonporous, and have such a race track shaped hole (two parallel lines joined on either end by semi-circles) through it (see FIG. 4A), then the coupling can be made nearly ideal. The reflector, may be designed with the ends of the race track fully enclosed except for holes for the laser rod and lamp on both ends, sized as well to help govern the water flow over the rod and lamp, and also a recess to support and position of the flow tube within, to make it concentric with the laser rod hole and laser rod. Enclosing the flow tube within such a reflector can be achieved by making reflector in two halves which are cemented together with the flow tube inside, or it can be achieved by using a one piece reflector with the race track hole clear through, and auxiliary reflecting end plates, having the required holes and recess, to achieve the same objective. In either case, the diffuse reflector encloses the pumping cavity to the fullest extent possible, for maximum efficiency.

In some embodiments, the inventive laser devices of the present invention can utilize diffuse reflector made by sintering or partially melting well-mixed powders consisting of high purity zirconium dioxide, ZrO2, (a material high index of refraction and ultra-low optical absorption, spanning the range of visible light and extending into the ultraviolet and infrared), with particle sizes in the 0.3 μm to 10 μm diameter range (preferably the 0.3 μm to 1 μm range) or in the nanoparticle range, together with other high-purity, transparent oxide powders, singularly or in combination, including but not limited to barium sulfate, BaSO4, borosilicate glass, 7056 alkali borosilicate (BS) glass (Corning), quartz, fused quartz, and fused silica, of sizes comparable to the above.

In the mixing of ZrO2 with BaSO4 as an example, particles of nominally the same size will result in substantial air gaps among the particles and the material will be porous in the bulk, surface glazing as is currently practiced widely, can be used to correct this. For example, the use of high purity nano-particles as one or the other of the materials, one of high index and the other of comparatively low index to minimize or eliminate the space between the particles of high and low index, making the material nonporous in the bulk.

For example, the dry mixture can be mixed and/or shake while turning it over sufficiently slowly so that gravity does not result in the larger material rising out of the mixture, prior to putting the mixture into a furnace to melt the low index component. In some embodiments, the size of the particle roughly corresponds to the wavelength of the light. In some embodiments, to reflect broadband, a range of sizes from 0.4 μm to 2 μm mixed together can be utilized. In some embodiments, particles are selected based on conditions that relatively small in diameter particles in comparison with the wavelength of the light may not efficiently scatter the light and too large particles may reduce the quantity or density of reflective surfaces in the reflector, which would necessitate a relatively larger thickness of reflector to be efficient as the inventive diffuse reflectors of the present invention.

For example, if ZrO2 particles are the larger particles, mixing of the components should be in such proportion to provide minimal separation between the ZrO2 particles, which remain solid and do not melt. The surfaces of the ZrO2 provide the bulk of the diffuse scattering. The other materials provide interstitial fill between the ZrO2 particles, which for nonporous reflectors would be sufficient to block coolant entry. Their index is sufficiently low that the efficiency of scatter from the interface is still substantial. For example, the index of BaSO4 is 1.636. The scattering efficiency is dependent on the square of the ratio of the index of refraction of the materials on either side of the interface. Thus, when comparing BaSO4 against air, this ratio is ≈1.75, meaning that one would need only 75% greater thickness to use a nonporous combination of ZrO2 and BaSO4. With other lower index oxides, including fused silica, and borosilicate glass, the additional thickness compared with BaSO4 is sufficiently less.

In some embodiments, the inventive diffuse reflectors of the present invention can be made by using nano-partcles of the high index component, and sufficiently sized particles (for the wavelength of the light) of the low index component, or vice versa, to produce sufficient diffuse reflection from a nonporous solid.

In some embodiments, the system of the present invention may utilize in combination with systems/methods/applications described in U.S. Pat. No. 8,494,012 and/or U.S. Published Application No. 2014/0276686.

In some embodiments, the present invention provides for a laser pump chamber, including: at least one laser gain medium, at least one excitation source, and at least one diffuse reflector to direct and redirect an emission from the excitation source into the laser gain medium, wherein the at least one diffuse reflector is made from a diffuse reflector material comprising at least one of: 1) white quartz and 2) BaSO4.

In some embodiments, the diffuse reflector material comprises the white quartz and the BaSO4. In some embodiments, the at least one laser gain medium includes at least one of Ce:Nd:YAG, Nd:YAG, and/or alexandarite.

In some embodiments, the present invention provides, including a step of utilizing the laser pump chamber of the present invention for of at least one of: 1) at least one medical procedure, and 2) at least one dermatological procedure. In some embodiments, the at least one dermatological procedure is at least one of: i) hair removal, ii) a tattoo removal, and iii) a skin resurfacing.

While a number of embodiments of the present invention have been described, it is understood that these embodiments are illustrative only, and not restrictive, and that many modifications may become apparent to those of ordinary skill in the art. Further still, the various steps may be carried out in any desired order (and any desired steps may be added and/or any desired steps may be eliminated).

What is claimed is:

1. A laser pump chamber, comprising:
at least one laser gain medium,
at least one excitation source, and
at least one diffuse reflector to direct and redirect an emission from the excitation source into the laser gain medium,
wherein the at least one diffuse reflector is made from a diffuse reflector material comprising at least one of:
1) white quartz and
2) BaSO4.

2. The laser pump chamber of claim 1, wherein the diffuse reflector material comprises the white quartz and the BaSO4.

3. The laser pump chamber of claim 1, wherein the at least one laser gain medium comprises Nd:YAG.

4. The laser pump chamber of claim 1, wherein the at least one laser gain medium comprises alexandarite.

5. The laser pump chamber of claim 1, wherein the at least one laser gain medium comprises Ce:Nd:YAG.

6. A method, comprising,
utilizing the laser pump chamber of claim 1 for of at least one of:
1) at least one medical procedure, and
2) at least one dermatological procedure.

7. The method of claim 4, wherein the at least one dermatological procedure is at least one of:
i) hair removal,
ii) a tattoo removal, and
iii) a skin resurfacing.
